(19) United States
(12) Patent Application Publication
(10) Pub. No.: US 2003/0162992 A1
(43) Pub. Date: Aug. 28, 2003

(54) Preparation of intermediates useful in the synthesis of antiviral nucleosides

(76) Inventors: Kyoichi A. Watanabe, Stone Mountain, GA (US); Jinfa Du, Lilburn, GA (US)

Correspondence Address: KING & SPALDING, 191 PEACHTREE STREET, N.E., ATLANTA, GA, 30303-1763, US

(21) Appl. No.: 10/318,982

(22) Filed: Dec. 12, 2002

Related U.S. Application Data
(60) Provisional application No. 60/341,378, filed on Dec. 14, 2001.

(51) Int. Cl. C07C 69/76; C07C 67/24

(52) U.S. Cl. 560/103; 560/210; 560/240

(57) ABSTRACT

The present invention is an efficient process for the manufacture of α-acyloxyacetaldehyde, a key intermediate in the synthesis of 1,3-oxathiolane and 1,3-dioxolane nucleosides.

The present application claims priority to U.S. Serial No. 60/341,378 filed on Dec. 14, 2001.

### FIELD OF THE INVENTION

This application is in the field of synthetic organic chemistry and is specifically an improved process for the synthesis of versatile intermediates, α-acyloxyacetaldehydes and their acetals, and their application to the synthesis of certain biologically active nucleoside.

### BACKGROUND OF THE INVENTION

Acquired immune deficiency syndrome (AIDS) is a catastrophic disease that has reached enormous proportions. From July 1998 through June 1999 a total of 47,083 AIDS cases were reported in the US alone. With more than 2.2 million deaths in 1998, HIV/AIDS has now become the fourth leading cause of mortality and its impact is set to increase. More than 16 million people have died of AIDS since the late 1970s, according to the agency.

AIDS first came to the attention of the US Center for Disease Control and Prevention (CDC) in 1981 when seemingly healthy homosexual men developed Kaposi's sarcoma (KS) and Pneumocystis carinii pneumonia (PCP), two diseases that were only known to affect immunodeficient patients. Subsequently, the causative agent of AIDS, a lymphoadenopathy-associated retrovirus, now known as human immunodeficiency virus (HIV) was isolated at the Pasteur Institute in Paris, and later confirmed independently at the US National Cancer Institute.

Another virus that causes serious human health problems is the hepatitis B virus (HBV). HBV is second only to tobacco as a cause of human cancer. The mechanism by which HBV induces cancer is unknown. It is postulated that it may directly trigger tumor development, or indirectly trigger tumor development through chronic inflammation, cirrhosis, and cell regeneration associated with the infection.

After a 2- to 6-month incubation period during which the host is typically unaware of the infection, HBV infection can lead to acute hepatitis and liver damage, resulting in abdominal pain, jaundice and elevated blood levels of certain enzymes. HBV can cause fulminant hepatitis, a rapidly progressing, often fatal form of the disease in which large sections of the liver are destroyed.

Patients typically recover from the acute phase of hepatitis B virus infection. In some patients, however, high levels of viral antigen persist in the blood for an extended, or indefinite, period, causing a chronic infection. Chronic infections can lead to chronic persistent hepatitis. Patients suffering from chronic persistent HBV are most common in developing countries. By mid-1991, there were approximately 225 million chronic carriers of HBV in Asia alone, and worldwide, almost 300 million carriers. Chronic persistent hepatitis can cause fatigue, liver cirrhosis, and hepatocellular carcinoma, a primary liver cancer.

In Western, industrialized countries, the high-risk group for HBV infection includes those in contact with 4BV carriers or their blood samples. The epidemiology of HBV is similar to that of HIV/AIDS, which is a reason why HBV infection is common among patients infected with HIV or suffering from AIDS. However, HBV is more contagious than HIV.

In 1985, it was reported that the synthetic nucleoside 3′-azido-3′-deoxythymidine (AZT, Zidovudine, Retrovir) inhibited the replication of HIV and became the first FDA-approved drug to be used in the fight against AIDS. Since then, a number of other synthetic nucleosides, including 2′,3′-dideoxyinosine (ddI), 2′,3′-dideoxycytidine (ddC), 2′,3′-dideoxy-2′,3′-didehydrothymidine (d4T), (−)-2′,3′-dideoxy-3′-thiacytidine (3TC), and (−)-carbocyclic 2′,3′-didehydro-2′,3′-dideoxyguanosine (carbovir) and its prodrug abacavir, have proven effective against HIV. After cellular phosphorylation to the 5′-triphosphate by cellular kinases, these synthetic nucleosides are incorporated into a growing strand of viral DNA, causing chain termination because they lack a 3′-hydroxyl group. They can also inhibit the viral enzyme reverse transcriptase.

The discovery that a the oxathiolane nucleoside BCH-189 possessed potent activity against human immunodeficiency virus (HIV) replication (Belleau B. et al., 5^{th} International Conference on AIDS, Montreal, Canada, Jun. 4-9, 1989, #T.C.O. 1) prompted Chu et al. to synthesize the chiral products (+)- and (−)-BCH-189 (Tetrahedron Lett., 1991, 32, 3791). The latter, lamivudine, otherwise known as 3TC or epivir, is currently used clinically in the treatment of both HIV infection and hepatitis B virus (HBV) infection. 3TC and interferon are currently the only FDA-approved drugs for the treatment of HBV infection. Viral resistance develops within 6 months of 3TC treatment in about 14% of patients.

It was later determined that the 5-fluorocytosine analogue, (−)-FTC, is more active than 3TC against HIV (Choi W. et al., J. Am. Chem. Soc., 1991, 113, 9377). More recently, the racemic form of FTC or Racivir has been discovered to show beneficial effects against HIV. (Schinazi R. F. et al., Antimicrobial Agents Chemotherapy 1992, 2423, U.S. Pat. Nos. 5,204,665, 5,210,085, 5,914,331, 5,639,814). β-(−)-Cis-2-hydroxymethyl-5-(5-fluorocytosin-1-yl)-1,3-oxathiolane (FTC) is currently in clinical trials for the treatment of HIV and separately for HBV by Triangle Pharmaceuticals, Inc. See Schinazi et al. (1992) Selective inhibition of human immunodeficiency viruses by racemates and enantiomers of cis-5-fluoro-1-[2-(hydroxymethyl)-1,3-oxathiolane-5-yl]cytosine. Antimicrob. Agents Chemother. 2423-2431; U.S. Pat. Nos. 5,210,085, 5,914,331, 5,814,639; WO 91/11186; WO 92/14743.

These nucleosides are manufactured by condensation of a silylated purine or pyrimidine base with a 1,3-oxathiolane intermediate. U.S. Pat. No. 5,204,466 discloses a process to condense a 1,3-oxathiolane with a silylated pyrimidine using tin chloride as a Lewis acid, which provides virtually complete β-stereoselectivity (see also Choi et al., loc. cit.). Several U.S. patents disclose processes for the preparation of 1,3-oxathiolane nucleosides via condensation of a 1,3-oxathiolane-2-carboxylic acid ester with a protected silylated base in the presence of a silicon-based Lewis acid, followed by reduction of the ester to the corresponding hydroxymethyl group to afford the final product (see U.S. Pat. Nos. 5,663,320, 5,693,787, 5,696,254, 5,744,596, 5,756,706, 5,864,164).

U.S. Pat. No. 5,272,151 discloses a process that uses a 2-O-protected-5-O-acylated-1,3-oxathiolane for the preparation of nucleosides by condensation with a silylated purine or pyrimidine base in the presence of a titanium catalyst.

U.S. Pat. Nos. 5,466,806, 5,538,975, and 5,618,820 disclose processes for preparing 1,3-oxathiolane nucleosides comprising coupling of a base to an intact sugar moiety.

U.S. Pat. No. 6,215,004 discloses a process for producing 1,3-oxathiolane nucleosides that includes condensing 2-O-protected-methyl-5-chloro-1,3-oxathiolane with a silylated 5-fluorocytosine without a Lewis acid catalyst.

In all cases the 1,3-oxathiolane ring is prepared in one of the following ways: (i) reaction of an aldehyde derived from a glyoxylate or glycolic acid with mercaptoacetic acid in toluene in the presence of p-toluenesulfonic acid to give 5-oxo-1,3-oxathiolane-2-carboxylic acid (Kraus J-L. et al., Synthesis, 1991, 1046); (ii) cyclization of anhydrous glyoxylates with 2-mercaptoacetaldehyde diethylacetal at reflux in toluene to give 5-ethoxy-1,3-oxathiolane lactone (U.S. Pat. No. 5,047,407); (iii) condensation of glyoxylic acid ester with mercaptoacetaldehyde (dimeric form) to give 5-hydroxy-1,3-oxathiolane-2-carboxylic ester or (iv) coupling of an acyloxyacetaldehyde with 2,5-dihydroxy-1,4-dithiane, the dimeric form of 2-mercaptoacetaldehyde to form a 2-(acyloxy)methyl-5-hydroxy-1,3-oxathiolane. The lactone, 5-oxo compound, has to be reduced to the corresponding lactol during the process to synthesize nucleosides. The 2-carboxylic acid or its ester also has to be reduced to the corresponding 2-hydroxymethyl derivatives with the borane-methylsulfide complex.

The key intermediate aldehyde can be prepared using several methods: (i) lead tetraacetate oxidation of 1,4-di-O-benzoyl meso-erythritol (Ohle M., Ber., 1941, 74′,291), 1,6-di-O-benzoyl D-mannitol (Hudson C. S. et al., J. Am. Chem. Soc., 1939, 61, 2432) or 1,5-di-O-benzoyl-D-arabitol (Haskins W. T. et. al., J. Am. Chem. Soc., 1943, 65, 1663); (ii) preparation of monoacylated ethylene glycol followed by oxidation to aldehyde (Sheikh E. Tetrahedron Lett., 1972, 257; Mancuso A. J. & Swern D. Synthesis, 1981, 165; Bauer M., J. Org. Chem., 1975, 40, 1990; Hanessian S. et al., Synthesis, 1981, 394); (iii) acylation of ethylene chlorohydrin followed by dimethylsulfoxide oxidation (Kornblum N. et al., J. Am. Chem. Soc., 1959, 81, 4113); (iv) acylation of 1,2-isopropylideneglycerol followed by deacetonation and periodate oxidation (Shao M-J. et al., Synthetic Commun., 1988, 18, 359; Hashiguchi S. et al., Heterocycles, 1986, 24, 2273); (v) lead tetraacetate oxidation (Wolf F. J. & Weijlard J. Org. Synth., Coll. Vol., 1963, 4, 124); (vi) ozonolysis of allyl or 3-methyl-2-buten-1-ol acylate (Chou T.-S. et al., J. Chin. Chem. Soc., 1997, 44, 299; Hambeck R. & Just G. Tetrahedron Lett., 1990, 31, 5445); (vii) and more recently, by acylation of 2-butene-1,4-diol followed by ozonolysis (Marshall J. A. et al. J. Org. Chem., 1998, 63, 5962). Also, U.S. Pat. No. 6,215,004 discloses a process to prepare acyloxyacetaldehyde diethylacetal by acylation of 2,2-diethoxyethanol.

α-Acyloxyacetaldehyde is the key intermediate not only for the synthesis of those oxathiolane and dioxolane nucleosides but also for the synthesis of other biologically active compounds, such as mescarine (Hopkins M. H. et al., J. Am. Chem. Soc., 1991, 113, 5354), oxetanocin (Hambalek R. & Just J., Tetrahedron Lett., 1990, 31, 5445), kallolide A (Marshall J. A. et al., J. Org. Chem., 1998, 63, 5962), (±)-kumausallene and (+)-epi-kumausallene (Grese T. S. et al., J. Org. Chem., 1993, 58, 2468), and 1,3-dioxolane nucleosides.

In light of the importance of oxathiolane and dioxolane nucleosides in antiviral therapy, it is an object of the present invention to provide an improved process for the manufacture of the critical intermediate, x-acyloxyacetaldehyde.

It is another object of the present invention to provide a process for the manufacture of α-acyl-oxyacetaldehyde that is facile and efficient.

It a further object of the present invention to provide a process for the manufacture of α-acyl-oxyacetaldehyde that does not require the use of lead.

It is yet another object of the present invention to provide a process for the manufacture of α-acyl-oxyacetaldehyde that does not require the use of oxidative or reductive conditions.

It is yet another object of the present invention to provide a process for the manufacture of α-acyl-oxyacetaldehyde that does not require the use of ozone.

It is yet another object of the present invention to provide a process for the manufacture of α-acyl-oxyacetaldehyde that does not require low-yielding steps such as monoacylation of ethylene glycol or selective acylation of sugar alcohol.

### SUMMARY OF THE INVENTION

The present invention is an efficient process for the manufacture of α-acyloxyacetaldehyde, a key intermediate in the synthesis of 1,3-oxathiolane and 1,3-dioxolane nucleosides. α-Acyloxyacetaldehyde can be cyclized with the appropriate cocyclizing agent to form an oxathiolane or dioxolane ring and then coupled with any desired purine or pyrimidine base to form the desired nucleoside. Examples of nucleoside analogs that can be made according to this process include BCH-189, 3TC, racemic or enantiomerically enriched FTC, β-D-dioxolanyl-2,6-diaminopurine (DAPD) and racemic or enantiomerically enriched 5-fluoro-cytosine-1,3-dioxolane (FDOC), from available precursors. Compounds made according to the present invention can also be used as synthetic intermediates for the preparation of a large variety of other biologically active compounds, including but not limited to mescarine, oxetanocin, kallolide A, (±)-kumausallene and (+)-epi-kumausallene, or their pharmaceutically acceptable salts or prodrugs, as well as additional derivatives obtained by functional group manipulations.

This process utilizes an inexpensive 2,2-dialkoxyethyl halide precursor. In one embodiment, a process for the manufacture of an a-acyloxyacetaldehyde of the formula is provided:

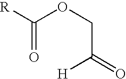

wherein R is hydrogen, alkyl (including but not limited to C₁₋₉ alkyl), alkenyl (including but not limited to C₂₋₉ alkenyl), alkynyl (including but not limited to C₂₋₉ alkynyl), or aryl (including but not limited to C₄₋₁₀ or C₆₋₁₀ aryl), that can be optionally substituted with one or more substituents that do not otherwise adversely affect the reaction process and wherein the R can be a chiral moiety; that includes the steps of:

a) reacting a 2,2-dialkoxyethyl halide of formula

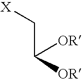

wherein X is a halide (F, Cl, Br, I), OTs, OMs or any other suitable leaving group;

R′ is independently an alkyl (including but not limited to C₁₋₉ alkyl), alkenyl (including but not limited to C₂₋₉ alkenyl), alkynyl (including but not limited to C₂₋₉ alkynyl), aryl (including but not limited to C₄₋₁₀ aryl or C₆₋₁₀ aryl), aralkyl, heteroaryl, or heterocycle;

with an appropriate carboxylate of formula ⁻OC(═O)R wherein R is hydrogen, alkyl (including but not limited to Cl S alkyl), alkenyl (including but not limited to C₂₋₉ alkenyl), alkynyl (including but not limited to C₂₋₉ alkynyl), or aryl (including but not limited to C₄₋₁₀ aryl or C₆₋₁₀ aryl), that can be optionally substituted with one or more substituents;

to obtain an acetal of the formula

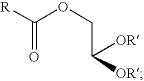

and

b) hydrolyzing the acetal to form the α-acyloxyacetaldehyde.

In one embodiment of the present invention, the α-acyloxyacetaldehyde can be further cyclized with mercaptoacetic acid; mercaptoacetaldehyde (dimeric form); mercaptoacetaldehyde dialkylacetal, such as diethylacetal; activated and/or protected mercaptoacetic acid or mercaptoacetaldehyde; or any other chemical equivalent of mercaptoacetic acid or mercaptoacetaldehyde to form a 1,3-oxathiolane, as illustrated below.

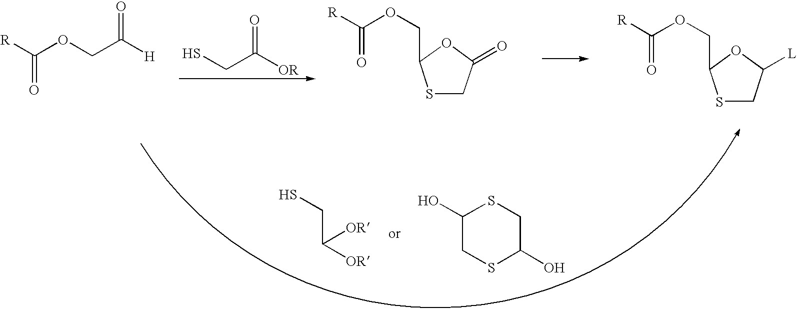

Wherein L is a leaving group, including, but not limited to O-acyl, O-alkyl, O-tosylate, O-mesylate, or halogen (Cl, Br, I, F); and R and R′ are as defined above.

In an alternate embodiment of the present invention, the α-acyloxyacetaldehyde can be further cyclized with glycolic acid; glycoaldehyde (dimeric form); glycoaldehyde dialkylacetal such as diethylacetal; activated and/or protected glycolic acid or glycoaldehyde; or any other chemical equivalent of glycolic acid or glycoaldehyde to form a 1,3-dioxolane, as illustrated below.

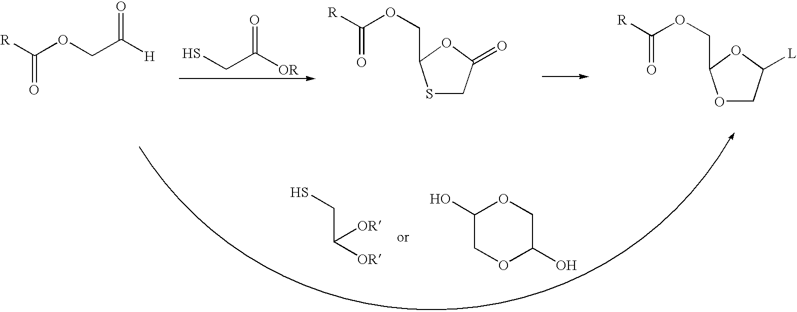

Wherein L is a leaving group, including, but not limited to O-acyl, O-alkyl, O-tosylate, O-mesylate, or halogen (Cl, Br, I, F); and R and R′ are as defined above.

In a further embodiment of the present invention, the 1,3-oxathiolane or 1,3-dioxolane can be further coupled, optionally in the presence of a Lewis acid such as BF₃.Et₂O, TMSCl, TMSI, TMSTf, SnCl₄ or TiCl₄, with a purine or pyrimidine base, including but not limited to cytosine, thymidine, uridine, guanine, adenine or inosine, optionally substituted as desired, with a moiety including, but not limited, to halogen (F, Cl, Br, I), such as 5-fluorocytosine, alkyl, alkenyl, alkynyl, cycloalkyl or acyl, to form a protected nucleoside, optionally followed by stereoselective or non-stereoselective deprotection.

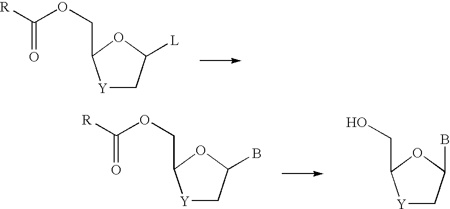

Y is O or S; B is a purine or pyrimidine or derivative thereof, as described herein.

In general, the R′ substituents are not particularly important to the reaction because they are hydrolyzed and removed during the formation of the α-acyloxyacetaldehyde. Therefore, the R′ substituent can be any moiety that does not otherwise interfere with the reaction.

In one embodiment, R is selected as a chiral moiety, which remains in the formed nucleoside in the ester at the 5′-position. The chiral R group is then suitably positioned to facilitate the separation of enantiomers via fractional crystallization, chiral or conventional chromatography, enzymatic resolution or the like. A number of chiral groups are known for this purpose, such as menthyl (L or D), norephedrine (D or L). In general, any chiral group that facilitates the separation of enantiomers will suffice. Preferred chiral R groups are those that have the chiral center in close proximity to the nucleoside.

In a particular embodiment of the present invention, the nucleoside is a β-D-nucleoside. In an alternate embodiment of the present invention, the nucleoside is a β-L-nucleoside.

### DETAILED DESCRIPTION OF THE INVENTION

The present invention is an efficient process for the manufacture of α-acyloxyacetaldehyde, the key intermediate for the synthesis of 1,3-oxathiolane and 1,3-dioxolane nucleosides, and in particular BCH-189, 3TC, racemic or enantiomerically enriched FTC, β-D-DAPD and racemic or enantiomerically enriched FDOC, from available precursors, that does not incorporate a low-yielding step, such as monoacylation of ethylene glycol or selective acylation of sugar alcohol, and does not require oxidation or reduction, rendering the process amenable to large-scale production. The a-acyloxyacetaldehyde can then be cyclized with an appropriate cocyclizing agent and coupled with a purine or pyrimidine base, as needed, by methods known in the art. Compounds made according to the present invention can also be used as synthetic intermediates for the preparation of a large variety of other biologically active compounds, including but not limited to mescarine, oxetanocin, kallolide A, (±)-kumausallene and (+)-epi-kumausallene, or their pharmaceutically acceptable salts or prodrugs, as well as additional derivatives obtained by functional group manipulations.

This process utilizes an inexpensive 2,2-dialkoxyethyl halide precursor. In one embodiment, a process for the manufacture of an α-acyloxyacetaldehyde of the formula below is provided:

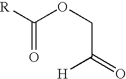

wherein R is hydrogen, alkyl (including but not limited to C₁₋₉ alkyl), alkenyl (including but not limited to C₂₋₉ alkenyl), alkynyl (including but not limited to C₂₋₉ alkynyl), or aryl (including but not limited to C₄₋₁₀ or C₆₋₁₀ aryl), that can be optionally substituted with one or more substituents that do not adversely affect the process and is optionally a chiral moiety; that includes the steps of:

a) reacting a 2,2-dialkoxyethyl halide of formula

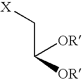

wherein X is a halide (F, Cl, Br, I), OTs, OMs or any other suitable leaving group and each R′ is independently an alkyl (including but not limited to C₁₋₉ alkyl); R′ is independently an alkyl (including but not limited to C₁₋₉ alkyl), alkenyl (including but not limited to C₂₋₉ alkenyl), alkynyl (including but not limited to C₂₋₉ alkynyl), aryl (including but not limited to C₄₋₁₀ aryl or C₆₋₁₀ aryl), aralkyl, heteroaryl, or heterocycle;

with an appropriate carboxylate of formula ⁻OC(═O)R wherein R is hydrogen, alkyl (including but not limited to C₁₋₉ alkyl), alkenyl (including but not limited to C₂₋₉ alkenyl), alkynyl (including but not limited to C₂₋₉ alkynyl), or aryl (including but not limited to C₄₋₁₀ or C₆₋₁₀ aryl), that can be optionally substituted with one or more substituents that do not otherwise adversely affect the process;

to obtain an acetal of the formula

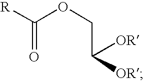

and

b) hydrolyzing the acetal to form the α-acyloxyacetaldehyde.

In one embodiment of the present invention, the α-acyloxyacetaldehyde can be further cyclized with mercaptoacetic acid; mercaptoacetaldehyde (dimeric form); mercaptoacetaldehyde dialkylacetal such as diethylacetal; activated and/or protected mercaptoacetic acid or mercaptoacetaldehyde; or any other chemical equivalent of mercaptoacetic acid or mercaptoacetaldehyde to form a 1,3-oxathiolane, as illustrated below.

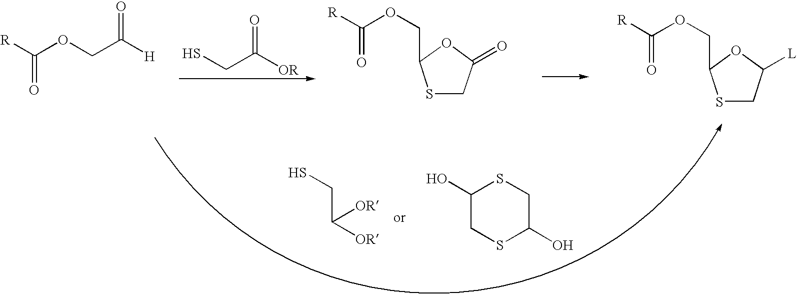

Wherein L is a leaving group, including, but not limited to O-acyl, O-alkyl, O-tosylate, O-mesylate, or halogen (Cl, Br, I, F); and R and R′ are as defined above.

In an alternate embodiment of the present invention, the (X-acyloxyacetaldehyde can be further cyclized with glycolic acid; glycoaldehyde (dimeric form); glycoaldehyde dialkylacetal such as diethylacetal; activated and/or protected glycolic acid or glycoaldehyde; or any other chemical equivalent of glycolic acid or glycoaldehyde to form a 1,3-dioxolane, as illustrated below.

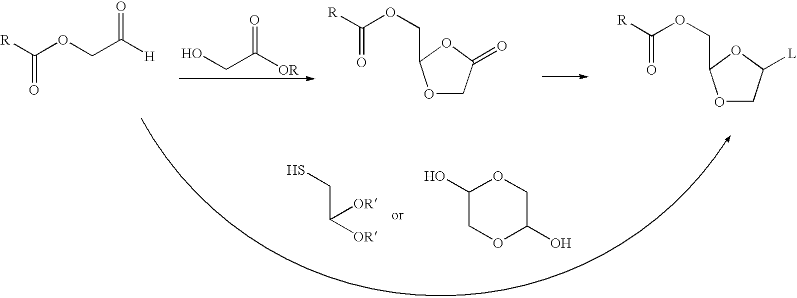

Wherein L is a leaving group, including, but not limited to O-acyl, O-alkyl, O-tosylate, O-mesylate, or halogen (Cl, Br, I, F); and R and R′ are same as above.

In a further embodiment of the present invention, the 1,3-oxathiolane or 1,3-dioxolane can be further coupled, optionally in the presence of a Lewis acid such as BF₃.Et₂O, TMSCl, TMSI, TMSTf, SnCl₄ or TiCl₄, with a purine or pyrimidine base, including but not limited to cytosine, thymidine, uridine, guanine, adenine or inosine, optionally substituted as desired, with a moiety including, but not limited, to halogen (F, Cl, Br or I) such as 5-fluorocytosine, alkyl, alkenyl, alkynyl, cycloalkyl or acyl, to form a protected nucleoside, optionally followed by stereoselective or non-stereoselective deprotection.

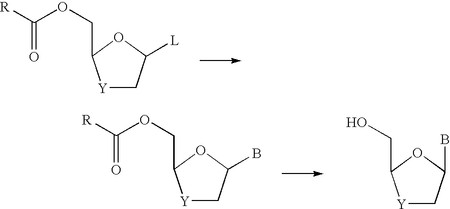

Y is O or S; B is a purine or pyrimidine or derivative thereof, as described herein.

I. Definitions

As used herein, the term “substantially free of,” “substantially in the absence of” or “isolated” refers to a nucleoside composition that includes at least 95%, and preferably 99% to 100% by weight, of the designated enantiomer of that nucleoside. In a preferred embodiment, the process produces compounds that are substantially free of enantiomers of the opposite configuration.

The term “alkyl,” as used herein, unless otherwise specified, refers to a saturated straight, branched, or cyclic, primary, secondary, or tertiary hydrocarbon. The term includes both substituted and unsubstituted alkyl groups. The alkyl group may be optionally substituted with any moiety that does not otherwise interfere with the reaction or that provides an improvement in the process, including but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene et al., Protective Groups in Organic Synthesis, John Wiley & Sons, Second Edition, 1991, hereby incorporated by reference.

In the text, whenever the term C(alkyl range) is used, the term independently includes each member of that class as if specifically and separately set out. As a nonlimiting example, the term “C₁₋₉” independently represents each species that falls within the scope. Alkyl groups include, but are not limited to the radicals of methane, ethane, propane, cyclopropane, 2-methylpropane (isobutane), n-butane, 2,2-dimethylpropane (neopentane), cytobutane, 1,1 dimethylcyclopropane, 2-methylbutane, trans-1,2-dimethylcyclopropane, ethylcyclopropane, n-pentane, methylcyclobutane, cis-1,2-dimethylcyclopropane, spiropentane, cyclopentane, 2,2-dimethylbutane, 1,1,2-trimethylcyclopropane, 2,3-dimethylbutane, 2-methylpentane, 3-methylpentane, 1,2,3-trimethylcyclopropane, n-hexane, ethylcyclobutane, methylcyclopentane, 2,2dimethylpentane, 2,4-dimethylpentane, cyclohexane, 2,2,3-trimethylbutane, 3,3-dimethylpentane, 1,1-dimethylcyclopentane, 2,3-dimethylpentane, 2-methylhexane, trans-1,3-dimethylcyclopentane, cis-1,3-dimethylcyclopentane, 3-methylhexane, trans-1,2-dimethylcyclopentane, 3-ethylpentane, quadricyclane (quadricyclo [2,2,1,0^{2.6},0^{3.5}]heptane), n-heptane, 2,2,4-trimethylpentane, cis-1,2-dimethylcyclopentane, methylcyclohexane, ethylcyclopentane, 1,1,3-trimethylcyclopentane, 2,2-dimethylhexane, 2,5-dimethylhexane, 1,trans-2,cis-4trimethylcyclopentane, 2,4-dimethylhexane, 2,2,3-trimethylpentane, 1,trans-2,cis-3-trimethylcyclopentane, 3,3-dimethylhexane, 2,3,4-trimethylpentane, 1,1,2-trimethylcyclopentane, 2,3,3-trimethylpentane, 2,3-dimethylhexane, 3-ethyl-2-methylpentane, 1,cis-2,trans-4-trimethylcyclopentane, 1,cis-2,trans-3trimethylcyclopentane, 2-methylheptane, 4-methylheptane, 3,4-dimethylhexane, 1,cis-2,cis-4trimethylcyclopentane, 3-ethyl-3-methylpentane, 3-ethylhexane, 3-methylheptane, cylotheptane (suberane), trans-1,4-dimethylcyclohexane, 1,1-dimethylcyclohexane, cis-1,3-dimethylcychohexane, trans-]-ethyl-3-methylcyclopentane, trans-1-ethyl-2-methylcyclopentane, cis-1-ethyl-3-methylcyclopentane, 1-ethyl-1-methylcyclopentane, 2,2,4,4-tetramethylpentane, 1,cis-2-cis-3-trimethylcyclopentane, trans-1,2-dimethylcyclohexane, 2,2,5-trimethylhexane, trans-1,3-dimethylcyclohexane, n-octane, isopropylcyclopentane, 2,2,4-trimethylhexane, cis-1-ethyl-2-methylcyclopentane, cis-1,2-dimethylcyclohexane, 2,4,4-trimethylhexane, n-propylcyclopentane, 2,3,5-trimethylhexane, ethylcyclohexane, 2,2-dimethylheptane, 2,2,3,4-tetramethylpentane, 2,4-dimethylheptane, methylcycloheptane, 2,2,3-trimethylhexane, 4-ethyl-2-methylhexane, 3-ethyl-2.2-dimethylpentane, 4,4-dimethylheptane, 2,6-dimethylheptane, 2,5-dimethylheptane, 3,5-dimethylheptane, bicyclo[4.2.0]octane, cis-bicyclo[3.3.0]octane, 2,4-dimethyl-3-ethylpentane, 1,1,3-trimethylcyclohexane, 3,3-dimethylheptane, 2,2,5,5-tetramethylhexane, 2,3,3-trimethylhexane 3-ethyl-2-methylhexane, trans-1,3,5-trimethylcyclohexane, 2,3,4-trimethylhexane, 1,3,5-trimethylcyclohexane, trans-1,2,4-trimethylcyclohexane, 2,2,3,3-tetramethylpentane, 4-ethyl-3-methylhexane, 3,3,4-trimethylhexane, 2,3-dimethylheptane, 3,4-dimethylheptane, 3-ethyl-3-methylhexane, 4-ethylheptane, 2,3,3,4-tetramethylpentane, 2,3-dimethyl-3-ethylpentane, trans-1,2,3-trimethylcyclohexane, 1-isopropyl-e-methylcyclopentane (pulegan), 4-methyloctane, 1-isopropyl-2-methylcyclopentane, 3-ethylheptane, 2-methyloctane, cis-1,2,3-trimethylcyclohexane, 3-methyloctane, 2,4,6-trimethylheptane, cis-1,2,4-trimethylcyclohexane, 3,3-diethylpentane, 2,2-dimethyl-4-ethylhexane, 2,2,4-trimethylheptane, 2,2,4,5-tetramethylhexane, 2,2,5-trimethylheptane, 2,2,6-trimethylheptane, 2,2,3,5-tetramethylhexane, nopinane (7,7-dimethylbicyclo[3.1.1]heptane), trans-1-ethyl-r-methylcyclohexane, cycloctane, 1-ethyl-2-methylcyclohexane, n-nonane, 1,3,3-trimethylbicyclo[2.2.1]heptane(fenchane), trans-1-ethyl-4-methylcyclohexane, cis-1,1,3,5-tetramethylcyclohexane, cis-1-ethyl-4-methylcyclohexane, 2,5,5-trimethylheptane, 2,4,4-trimethylheptane, 2,3,3,5-tetramethylhexane, 2,2,4,4-tetramethylhexane, isopropylcyclohexane, 1,1,2,2-tetramethylcyclohexane, 2,2,3,4-tetramethylhexane, 2,2-dimethyloctane, 3-ethyl-2,2,4-trimethylpentane, 3,3,5-trimethylheptane, 2,3,5-trimethylheptane, 2,4-dimethyloctane, d,1-cis-1-ethyl-3-methylcyclohexane, d,1-2,5-dimethyloctane, 1,1,3,5-tetramethylcyclohexane, n-butylcyclopentane, n-propylcyclohexane, 2,3,5-trimethylheptane, 2,5-dimethyl-3-ethylhexane, 2,4,5-trimethylheptane, 2,4-dimethyl-3-isopropylpentane, 2,2,3-trimethylheptane, 2,4-dimethyl-4-ethylhexane, 2,2-dimethyl-3-ethylhexane, 2,2,3,4,4-pentamethylpentane, 1,1,3,4-tetramethylcyclohexane, 5-ethyl-2-methylheptane, 2,7-dimethyloctane, 3,6-dimethyloctane, 3,5-dimethyloctane, 4-isopropylheptane, 2,3,3-trimethylheptane, 4-ethyl-2-methylheptane, 2,6-dimethyloctane, 2,2,3,3-tetramethylhexane, trans-1-isopropyl-4-methylcyclohexane(p-menthane), 4,4-dimethyloctane, 2,3,4,5-tetramethylhexane, 5-ethyl-e-methylheptane, 3,3-dimethyloctne, 4,5-dimethyloctane, 3,4-diethylhexane, 4-propylheptane, 1,1,4-trimethylcycloheptane (eucarvane), trans-1,2,3,5-tetramethylcyclohexane, 2,3,4,4-tetramethylhexane, 2,3,4-trimethylheptane, 3-isopropyl-2-methylhexane, 2,2,7-trimethylbicyclo[2.2.1]heptane(a-frenchane), 3-methylheptane, 2,4-dimethyl-3-ethylhexane, 3,4,4-trimethylheptane, 3,3,4-trimethylheptane, 3,4,5-trimethylheptane, 2,3-dimethyl-4-ethylhexane, 1-methyl-e-propylcyclohexane, 2,3-dimethyloctane, d,1-pinane, 2,3,3,4-tetramethylhexane, 3,3-dimethyl-4-ethylhexane, 5-methylnonane, 4-methylnonane, 3-ethyl-2-methylheptane, d,1-1-isopropyl-3-methylcyclohexane(d,1-menthane), 2,2,3,3,4-pentamethylpentane, trans-1,2,4,5-tetramethylcyclohexane, 3,3-diethylhexane, 2-methylnonane, d-1-isopropyl-3-methylcyclohexane (d-m-menthane), 3-ethyl-4-methylheptane 4-ethyl-3-methylheptane, 4-ethyl-4-methylheptane, 1-β-pinane, 3-methylnonane, 3-ethyloctane, 4-ethyloctane, 3-ethyl-2,2,3-trimethylpentane, I-1-isopropyl-3-methylcyclohexane (1-m-menthane)cis-1-isopropyl-4-methylcyclohexane (cis-p-menthane), cis-1,2,3,5-tetramethylcyclohexane, 2,3-dimethyl-3-ethylhexane, 1-isopropyl-4-methylcyclohexane (p-menthane), 3,4-dimethyl-3-ethylhexane, 3,3,4,4-tetramethylhexane, cyclononane, 1-isopropyl-2-methylcyclohexane (o-menthane), cis-1,2,4,5-tetramethylcyclohexane, 1-methyl-1-propylcyclohexane, 1-methyl-4-propylcyclohexane, 1-methyl-2-propylcyclohexane, n-pentrylcyclopentane, n-butylcyclohexane and isoamylcyclohexane. It is understood to those of ordinary skill in the art that the relevant alkyl radical is named by replacing the suffix “-ane” with the suffix “-yl”.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid or phosphonate, either unprotected, or protected as necessary, as known to those skilled in the art, for example, as taught in Greene et al., Protective Groups in Organic Synthesis, John Wiley & Sons, Second Edition, 1991, hereby incorporated by reference. Non-limiting examples of alkenyl groups include methylene, ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group may be optionally substituted with any moiety that does not adversely affect the reaction process, including but not limited to hydroxyl, halo (including independently F, Cl, Br, and I), perfluoro alkyl including trifluoromethyl, amino, alkylamino, arylamino, alkoxy, aryloxy, nitro, cyano, acyl, amido, carboxamido, carboxylate, thiol, alkylthio, azido, sulfonic acid, sulfate, phosphonic acid, phosphate, or phosphonate, either unprotected, or protected as necessary, as known to those skilled in the art, for example as taught in Greene, et al., Protective Groups in Organic Synthesis, John Wiley and Sons, Second Edition, 1991, hereby incorporated by reference. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “aryl”, alone or in combination, means a carbocyclic aromatic system containing one, two or three rings wherein such rings may be attached together in a pendent manner or may be fused. Non-limiting examples of aryl include phenyl, or the following aromatic group that remains after the removal of a hydrogen from the aromatic ring: benzene, toluene, ethylbenzene, 1,4-xylene, 1,3-xylene, 1,2-xylene, isopropylbenzene (cumene), n-propylbenzene, 1-ethyl-3-methylbenzene (m-ethyltoluene), 1-ethyl-4-methylbenzene (p-ethyltoluene), 1,3,5-trimethylbenzene (mesitylene), 1-ethyl-2-methylbenzene (o-ethyltoluene), tert-butylbenzene, 1,2,4-trimethylbenzene (pseudodocumene), isobutylbenzene, sec-butylbenzene, 3-isopropyl-methylbenzene (3-isopropyltoluene; m-cymene), 1,2,3-trimethylbenzene (hemimellitene), trans-propenylbenzene, indane, 4-isopropyl-1-methylbenzene (4-isopropyltoluene; 4-cymene), 2-isopropyl-methylbenzene (2-isopropyltoluene; 2-cymene), 1,3-diethbenzene, 1 methyl-3-proplybenzene (m-propyltoluene), indene, n-butylbenzene, 1-methyl-4-propylbenzene (p-propyltoluene), 1,2-diethylbenzene, 1,4-diethylbenzene, 1,3-dimethyl-5-ethylbenzene, 1-methyl-2-propylbenzene (o-propyltoluene), 2,2-dimethyl-1-phenylpropane (neopentylbenzene), 1,4-dimethyl-2-ethylbenzene, 2-methylindane, 3-methyl-2-phenylbutane, 1-methylindane, 1,3-dimethyl-4-ethylbenzene, 3-tert-butyl-menthylbenzene, (3-tert-butyltoluene), 1,2-dimethyl-4-ethylbenzene, 1,3-dimethyl-2-ethylbenzene, 3-phenylpentane, 1-ethyl-3-isopropylbenzene, 2-methyl-2-phenylbutane, 4-tert-butyl 1-methylbenzene (4-tert-butyltoluene), 1-ethyl-2-isopropylbenzene, 2-phenylpentane, 1,2-dimethyl-3-ethybenzene, 3-sec-butyl-1-methylbenzene, (3-sec-butylotoluene), 3-isobutyl-1-methylbenzene, (3-isobutyltoluene), d-2-methyl-1-phenylbutane, 1,3-dimethyl-5-isopropyl-benzene, 2-phenyl-cis-2-butene, 4-isobutyl-methylblenzene (p-isobutyltoluene), 2-sec-butyl-1-methylbenzene (2-sec-butyltoluene), 2-isobutyl-1-methylblenzene (o-isobutyltoluene), 1,4-dimethyl-2-isopropyl-benzene, 1-ethyl-4-isopropylbenzene, d,1-2-methyl-1-phenylbutane, 1,2,3,5-tetramethylbenzene (isodurene), 3-methyl-1-phenylbutane (isopentylbenzene), 1,3-dimethyl-2-isopropylbenzene, 1,3-dimethyl-4-isopropylbenzene), 3-methylindene, 4-sec-butyl-1-methylbenzene (p-sec-butyltoluene), 2-tert-butyl-1-methylbenzene (2-tert-butyltoluene), 3,5-diethyl-1-methylbenzene (3,5-diethyltoluene), 2-butyl-1-methylbenzene (2 butyltoluene), 1-ethyl-3-propylbenzene, 1,2-dimethyl-4-isopropylbenzene, 1,2-dimethyl-3-isopropylbenzene, 1-ethyl-2-propylbenzene, 1,3-di-isopropyllbenzene, 1,2-diethyl-4-methylbenzene, 1,2-di-isopropylbenzene, 1,4-dimethyl-2-proplybenzene, 1,2,3,4-tetramethylbenzene (prehnitene), 1-ethyl-4-propylbenzene, 3-butyl-1-methlybenzene (m-butyltoluene), 2,4-diethyl-1-methylbenzene (2,4-diethyltoluene), n-pentylbenzene, 3-methyl-3-phenylpentane, 1,3-dimethyl-5-tert-butylbenzene, 1,3-dimethyl-4-propylbenzene, 1,2-diethyl-3-methylbenzene, 4-butyl-1-methylbenzene, 4-butyl-1-methylbenzene, 1,2,3,4-tetrahydronaphthalene, 1,3-diethyl-2-propylbenzene, 2,6-diethyl-1-methylbenzene, 1,2-dimethyl-4-propylbenzene, 1,3-dimethyl-5-propylbenzene, 2-methyl-3-phenylpentane, 4-teri-butyl-1,3-dimethylbenzene, 1,4-di-isopropylbenzene, 1,2-dimethyl-3-propylbenzene, 1-teri-butyl-4-ethylbenzene, d,1-3-phenylhexane, 2-ethyly-1,3,5-trimethyl-benzene, 3-ethyly-4-isopropyl-1-methylbenzene, 5-ethyl-1,2,4-trimethylbenzene, 6-ethyl-1-2,4-trimethylbenzene, 2-phenylhexane, 2-methyl-1-phenylpentane, 4-isopropyl-1-propylbenzene, 1,3-dipropylbenzene, 5-ethyl-1,2,3-trimethylbenzene, 1,2,4-triethylbenzene, 1,3,5-triethylbenzene, 2-methyl-1,2,3,4-tetrahydronaphthalene, 1-methyl-1,2,3,4-tetrahydronaphthalene, 4-ethyl-1,2,3-trimethylbenzene, 1,4-dipropylbenzene, 3-methyl-1-phenylpentane, 2-propyl-1,3,5-trimethylbenzene, 1,1-dimethyl-1,2,3,4-tetrahydronaphthalene, 3-tert-butyl-1-isopropylbenzene, 1-methyl-3-pentylbenzene, 4-tert-butyl-1-isopropylbenzene, 2-methyl-2-phenylhexane, 2,4-di-isopropyl-1-methylbenzene, 3-methyl-3-phenylhexane, n-hexylbenzene, 3-phenylheptane, 2,6-di-isopropyl-1-methylbenzene, 5-propyl-1,2,4-trimethylbenzene, 6-methyl-1,2,3,4-tetrahydronaphthalene, 2,2-dimethyl-1,2,3,4-tetrahydronaphthalene, 2-phenylheptane, 5-methyl-1,2,3,4-tetrahydronaphthalene, 2-ethyl-1,2,3,4-tetrahydronaphthalene, cyclohexylbenzene, 1-ethyl-1,2,3,4-tetrahydronaphthalene, 2,5-dimethyl-1,2,3,4-tetrahydronaphthalene, 2,8-dimethyl-1,2,3,4-tetrahydronaphthalene, 2,7-dimethyl-1,2,3,4-tetrahydronaphthalene, 2,6-dimethyl-1,2,3,4-tetrahydronaphthalene, 1,4-di-sec-butylbenzene, 1,5-dimethyl-1,2,3,4-tetrahydronaphthalene, 3-ethyl-3-phenylhexane, 6-ethyl-1,2,3,4-tetrahydronaphthalene, 2-methyl-1-phenyl-1-butene, 5-ethyl-1,2,3,4-tetrahydronaphthalene, n-heptylbenzene, 1-methylnaphthalene, 5,6-dimethyl-1,2,3,4-tetrahydronaphthalene, 6,7-dimethyl-1,2,3,4-tetrahydronaphthalene, 5,7-dimethyl-1,2,3,4-tetrahydronaphthalene, 2-ethylnaphthalene, 1-7-dimethylnaphthalene, 1,6-dimethylnaphthalene, 1,3-dimethylnaphthalene, n-octylbenzene, 1-allylnaphthalene, 1-isopropylnaphthalene, 1,4-dimethylnaphthalene, 1,1-diphenylethane, 2-isopropylnaphthalene, 2-propylnaphthalene, 1-propylnaphthalene, 1,3,7-trimethylnaphthalene, 1-isopropyl-7-methylnaphthalene, n-nonylbenzene, 2-butylnaphthalene, 2-tert-butylnaphthalene, 1-tert-butylnaphthalene, 1-butylnaphthalene, 4,5-benzindane, n-decylbenzene, 1-pentylnaphthalene, 2-pentylnaphthalene, n-undecylbenzene, 1-hexylnaphthalene, 2-hexylnaphthalene, n-dodecylbenzene, 1-heptylnaphthalene, 2-heptylnaphthalene, tridecylbenzene, 1-octylnaphthalene, 2-octylnaphthalene, 1-nonylnaphthalene, 2-nonylnaphthalene, 1-decylnaphthalene, 1,2,6-trimethylnaphthalene, diphenylmethane, 1,2,3-trimethylnaphthalene, 1,6,7-trimethylnaphthalene, 2-isopropylazulene, 1,4-dimethyl-7-isopropylazulene, 2,6-dimethylphenanthrene, 1,2,5-trimethylnaphthalene, 1-propylphenanthrene, 5-isopropylazulene, 5-isopropylazulene, 2-propylphenanthrene, 2-methylnaphthalene, 1-ethyl-5-methylnaphthalene, 9-isopropylnaphthalene, 6-isopropylazulene, 2-ethyl-6-methylnaphthalene, 2-isopropylphenanthrene, 6-isopropyl-1-methylphenanthrene, 2-ethylazulene, 2,5,-dimethylphenanthrene, 1,3,5-trimethylnaphthalene, 3-ethyl-6-methylphenanthrene, 2-methylazulene, 1,3,8-trimethylnaphthalene, 4-methylphenanthrene, 1,4-dimethylphenanthrene, bibenzyl, methylenefluorene, 3,5-dimethylphenanthrene, 1,3-dimethylazulene, 7-methyl-3,4-benzphenanthrene, pentamethylbenzene, 1,2,4-trimethylnaphthalene, 3,3-dimethylstilbene, 1,4,5,7-tetramethylnaphthalene, 1,2,4,8-tetramethylnaphthalene 2,9-dimethylphenanthrene, 1,5-dimethylphenanthrene, 2-benzylnaphthalene, 1-benzylnaphthalene, 1-benzylnaphthalene, 1,2 dimethylazulene, 9-propylphenanthrene, 1,7-dimethyl-4-isopropylnaphthalene, 3-methylphenanthrene, 3,4-dimethylphenanthrene, 1-ethylphenanthrene, sym-diphenylacetylene, 9-ethylphenanthrene, 1,4,5-trimethylnaphthalene, 4-methylfluorene, 1,4,6,7-tretramethylnaphthalene, 1,2,3-trimethylphenanthrene, 1,8-dimethylnaphthalene, 8-methyl-3,4-benzphenanthrene, 2-ethylphenanthrene, 3,4-benzphenanthrene, 1,3,7-trimethylphenanthrene, 4-isopropyl-1-methylphenanthrene, 4,8-dimethylazulene, biphenyl, 2-methyl-3,4-benzphenanthrene, 3-methylpyrene, 1,4,7-trimethylphenanthrene, 1,4-dimethylanthracene, 4,9-dimethyl-1,2-benzanthracene, benzalfluorene, 1,3-dimethylphenanthrene, 1-methyl-3,4-benzphenanthrene, 3-isopropyl-1-methylphenanthrene, 1,2-binaphthyl, 2,3-dimethylphenanthrene, 1-ethyl-2-methylphenanthrene, 1,5-dimethylnaphthalene, 6-methyl-3,4-benzphenanthrene, naphthalene, 1,3,6,8-tetramethylnaphthalene, 1-ethyl-7methylphenanthrene, 9-methylanthracene, 1-isopropyl-7-methylphenanthrene, 6-methylazulene, 1,3-dimethylanthracene, 2,2-dimethylstilbene, 1-methylanthracene, 1,7-dimethylphenanthrene, 1,6-diphenylnaphthalene, 1,6-dimethylphenanthrene, 1,9-dimethylphenanthrene, 9-methylphenanthrene, 1,2,10-trimethylanthracene, 7-ethyl-1-methylphenanthrene, triphenylmethane, 5-isopropylnaphthanthracene, 3,9-dimethyl-1,2-benzanthracene, 5,6-benzindane, 12-isopropylnaphthanthracene, acenaphthene, 2,7-dimethylnaphthalene, 7-isopropyl-1-methylfluorene, azulene, retene, phenanthrene, 2,7-dimethfylphenanthrene, 2,3,6-trimethfylnaphthalene, 2-phenylnaphthalene, 1,2,3,4-tetrahydroanthracene, 2,3-dimethylnaphthalene, ethylidenefluorene, 1,7-dimethylfuorene, 1,1-dinaphthylmethane, fluoranthrene, 2,6-dimethylnaphthalene, 2,4-dimethylphenanthrene, fluorene, 4,10-dimethyl-1,2-benzanthracene, 4h-cyclopenta(def)phenanthrene, 1,3,8-trimethylphenanthrene, 11-methylnaphthanthracene, 5-methylchrysene, 1,2,5,6-tetramethylnaphthalene, cyclohept(fg)acenaphthene, 1,2,7-trimethylphenanthrene, 1,10-dimethyl-1,2-dibenzanthracene, 9,10-dimethyl-1,2-benzanthracene, benz(bc)aceanthrylene, 1-methylphenanthrene, 1,6,7-trimethylphenanthrene, 1,1-diacenaphthene, trans-stilbene, 3,4-benzflurorene, 9-isopropylnaphthanthracene, 6-methylnaphthanthracene, 5,8-dimethyl-1,2-bezanthracene, 8-isopropylnaphthanthracene, 1,4,5,8-tetramethylnaphthalene, 12-methylnaphthanthracene, 2-methyl-1,2-benzpyrene, 1,5-dimethylanthracene, 7-methylnaphthanthracene, 3,6-dimethylphenanthrene, 5-methyl-3,4-benzphenanthrene, 1,4-dimethylchrysene, 1,2-dimethylphenanthrene, 8,10-dimethyl-1,2-benzanthracene, 1,2,8-trimethylphenanthrene, 3-methyl-1,2-benzpyrene, 9-methyl-1-,2-benzpyrene, 9-phenylfluorene, 2-methylnaphthanthracene, pyrene, 9-methylnaphthanthracene, 4-methylchrysene, trans-trans-1,4-diphenyl-1,3-butadiene, cinnamalfluorene, 5-methylnaphthanthracene, 1,2-benzanthracene, 8-methylnaphthanthracene, 1,1-binaphthyl, di-1-naphthastibene, 6-methylchrysene, 3-methylnaphthanthracene, 2,6-dimethyl-1,2-benzanthracene, cyclopentadienophenanthrene, 10,11-benzfluoranthene, hexamethylbenzene, 3-methylchrysene, cholanthrene, 6-methyl-1,2-benzpyrene, 6,7-dimethyl-1,2-benzanthracene, 1,2-benzpyrene, 5,10-dimethyl-1,2-benzanthracene, 4,5-benzpyrene, 9,10-dimethylanthracene, 10-methylnaphthanthracene, 5,6-dimethyl-1,2-benzanthracene, 2,2-binaphthyl, 1,2-benfluorene, 1,8-dimethylphenanthrene, 8-methyl-1,2-benzpyrene, bifluorenylidene, 1,2,7,8-dibenzanthracene, 4-methylnaphthanthracene, 1,2,3,4-dibenzanthracene, di-2-fluorenylmethane, 2,3-benzfluorene, 5-methyl-1,2-benzpyrene, anthracene, 11,12-benzfluoranthene, 4-methyl-1,2-benzpyrene, 2,8-dimethylchrysene, 2-methylchrysene, 6,12-dimethylchrysene, 1,2-benzphenanthrene, di-2-naphthastilbene, 1-methylchrysene, 2,3,6,7-dibenzphenanthrene, 2,3,5,6-dibenzphenanthrene, 1,2,5,6-dibenzanthracene, perylene, picene, 1,2,3,4,5,6,7,8-tetrabenzanthracene, coronene. The term aryl includes both substituted and unsubstituted moieties. The aryl group may be optionally substituted with any moiety that does not adversely affect the process, including but not limited to halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Non-limiting examples of aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroayloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes fluoro, chloro, bromo and iodo.

The term “heteroatom,” as used herein, refers to oxygen, sulfur, nitrogen and phosphorus.

The term “acyl” refers to a carboxylic acid ester in which the non-carbonyl moiety of the ester group is any group that does adversely affect the process or that provides an advantageous effect. Nonlimiting examples are selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, alkyl or alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “purine base” or “pyrimidine base” includes, but is not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, pyrazolopyrimidinyl, guanine, adenine, hypoxanthine, 2,6-diaminopurine, 2-(Br, Fl, Cl or I)-purine optionally with a substituent including an amino or carbonyl group in the 6-position, and 6-(Br, Cl, or I)-purine optionally with, a substituent including an amino or carbonyl group in the 2-position. Functional oxygen and nitrogen groups on the base can be protected as necessary or desired. Suitable protecting groups are well known to those skilled in the a and include trimethylsilyl dimethylhexylsilyl, t-butyldimethylsilyl, and t-butyldiphenylsilyl, trityl, alkyl groups, and acyl groups such as acetyl and propionyl, methanesulfonyl, and p-toluenesulfonyl.

The terms “heteroaryl” or “heteroaromatic,” as used herein, refer to an aromatic that includes at least one sulfur, oxygen, nitrogen or phosphorus in the aromatic ring.

The term “heterocyclic” refers to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to those described above for aryl.

The term “chiral” refers to any carbon center in which the carbon atom is attached to four different substituents. The chiral group can be in the D or L configuration. Non-limiting examples of chiral moieties include menthyl, norephedrine, 2-octanyl, ethyl-3-hydroxybutyrate, ethyl-4-chloro-3-hydroxybutyrate, ethyl-4-chloro-3-hydroxybutyrate, ethyl-2-hydroxy-4-phenylbutyrate, 2-(1-hydroxyethyl)-pyridine, methyl-3-hydroxybutyrate, ethyl-3-hydroxybutyrate, 2-hydroxy-4-phenyl-butyric acid, 1-(3,4-methylenedioxy-phenyl)-2-propanol, 6-methyl-5-heptene-2-ol, 1-(2-naphthyl)-ethanol, trans-4-phenyl-3-butene-2-ol, 1-phenylethanol, 1-phenylethanol, 1-phenyl-2-propanol, 4-phenyl-2-butanol, ethyl-lactate, 4-cyanophenyl phenyl methanol chiral dichlorophthlate, 4-cyanophenyl phenyl methanol chiral dichlorophthlate, 4-bromophenyl phenyl methanol chiral dichlorophthlate, 4-bromophenyl phenyl methanol chiral dichlorophthlate, 4-methoxyphenyl phenyl methanol chiral dichlorophthlate, 4-methoxyphenyl phenyl methanol chiral dichlorophthlate, 4-chlorophenyl phenyl methanol chiral dichlorophthlate, 4-chlorophenyl phenyl methanol chiral dichlorophthlate, 4-nitrophenyl phenyl methanol chiral dichlorophthlate, 4-nitrophenyl phenyl methanol chiral dichlorophthlate, (4-bromophenyl)-(4-methylphenyl) methanol chiral dichlorophthlate, (4-bromophenyl)-(4-methylphenyl) methanol chiral dichlorophthlate, (4-bromophenyl)-phenyl-d5 methanol chiral dichlorophthlate, (4-bromophenyl)-phenyl-d5 methanol chiral dichlorophthlate, and chiraldichlorophthalic alcohol.

II. Stereochemistry

The nucleosides formed from these coupling reactions may have asymmetric centers and occur as racemates, racemic mixtures, individual diastereomers or enantiomers, with all isomeric forms being included in the present invention. Nucleosides having a chiral center may exist in and be isolated in optically active and racemic forms. Some compounds may exhibit polymorphism. The nucleosides formed from the coupling reaction can encompass racemic, optically-active, polymorphic or stereoisomeric form, or mixtures thereof, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

In one embodiment, R is selected as a chiral moiety, which remains in the formed nucleoside in the ester at the 5′-position. The chiral R group is then suitably positioned to provide for the separation of enantiomers via fractional crystallization, chiral or conventional chromatography, enzymatic resolution or the like. Optically active forms of the compounds can be prepared using any method known in the art, including by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique, whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through. Chiral chromatography, including simulated moving bed chromatography, is used in one embodiment. A wide variety of chiral stationary phases are commercially available.

III. Detailed Description of Process Steps

The key starting material for this process is an appropriate 2,2-dialkoxyethyl halide of formula

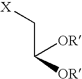

wherein X is a halide (F, Cl, Br or I) and each R′ is independently an alkyl (including but not limited to C₁₋₉ alkyl), alkenyl (including but not limited to C₂₋₉ alkenyl), alkynyl (including but not limited to C₂₋₉ alkynyl), aryl (including but not limited to C₄₋₁₀ aryl or C₆₋₁₀ aryl), aralkyl, heteroaryl, or heterocycle.

In an alternate embodiment, X is OTs, OMs or any other suitable-leaving group. The 2,2-dialkoxyethyl halide can be purchased or can be prepared by any known means including standard substitution and/or addition techniques. Since 2,2-dialkoxyethyl halides are inexpensive, in one embodiment the 2,2-dialkoxyethyl halide is purchased.

The 2,2-dialkoxyethyl halide can then be reacted with an appropriate carboxylate of formula ⁻OC(═O)R wherein R is hydrogen, alkyl (including but not limited to C₁₋₉ alkyl), C₂₋₉ alkenyl, alkynyl (including but not limited to C₂₋₉ alkynyl), or aryl (including but not limited to C₄₋₁₀ or C₆₋₁₀ aryl), that can be optionally substituted with one or more substituents. The carboxylate can be purchased or can be prepared by any known means, including reacting the corresponding carboxylic acid with a suitable base to obtain an alkali or alkaline-earth metal salt of carboxylic acid. The reaction can be carried out in a compatible solvent at a suitable temperature to yield the corresponding an acetal.

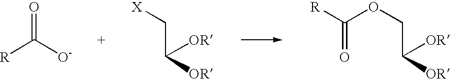

The acetal formation can be carried out in any reaction solvent that can achieve the necessary temperature and that can solubilize the reaction components. Nonlimiting examples are any aprotic solvent including, but not limiting to, alkyl solvents such as hexane and cyclohexane, toluene, acetone, ethyl acetate, dithianes, THF, dioxane, acetonitrile, dichloromethane, dichloroethane, diethyl ether, pyridine, N,N-dimethylformamide (DMF), dimethylsulfoxide (DMSO), dimethylacetamide, hexamethylphosphoric triamide or any combination thereof. In one embodiment, the solvent is a polar aprotic solvent, such as acetonitrile, DMF, DMSO or hexamethylphosphoric triamide, though preferably DMF.

The acetal formation can be carried out at any temperature that achieves the desired results, i.e., that is suitable for the reaction to proceed at an acceptable rate without promoting decomposition or excessive side products. Preferred temperatures are refluxing conditions, for example 153° C. for refluxing DMF.

Then hydrolysis of the acetal to yield the α-acyloxyacetaldehyde can be achieved using any, suitable organic or inorganic acid. For example, the hydrolysis can be promoted with aqueous formic acid.

This reaction can be accomplished at any temperature that allows the reaction to proceed at an acceptable rate without promoting decomposition or excessive side products. The preferred temperature is room temperature.

Appropriate solvents include any protic or aprotic solvent including, but not limiting to, alkyl solvents such as hexane and cyclohexane, toluene, acetone, ethyl acetate, dithianes, THF, dioxane, acetonitrile, dichloromethane, dichloroethane, diethyl ether, pyridine, dimethylformamide (DMF), dimethylsulfoxide (DMSO), dimethylacetamide, or any combination thereof, preferably THF.

The α-acyloxyacetaldehyde can then be cyclized to form a 1,3-oxathiolane ring or a 1,3-dioxolane ring, by known methods. For example, the 1,3-oxathiolane ring can be prepared in one of the following ways: (i) reaction of an aldehyde derived from a glyoxylate or glycolic acid with mercaptoacetic acid in toluene in the presence of p-toluenesulfonic acid to give 5-oxo-1,3-oxathiolane-2-carboxylic acid (Kraus, J-L. et al., Synthesis, 1991, 1046); (ii) cyclization of anhydrous glyoxylates with 2-mercaptoacetaldehyde diethylacetal at reflux in toluene to give 5-ethoxy-1,3-oxathiolane lactone (U.S. Pat. No. 5,047,407); (iii) condensation of glyoxylic acid ester with mercaptoacetaldehyde (dimeric form) to give 5-hydroxy-1,3-oxathiolane-2-carboxylic ester or (iv) coupling of an acyloxyacetaldehyde with 2,5-dihydroxy-1,4-dithiane, the dimeric form of 2-mercaptoacetaldehyde to form a 2-(ayloxy)methyl-5-hydroxy-1,3-oxathiolane. The lactone, 5-oxo compound, has to be reduced to the corresponding lactol during the process to synthesize nucleosides. The 2-carboxylic acid or its ester also has to-be reduced to the corresponding 2-hydroxymethyl derivatives with borane-methylsulfide complex. The 1,3-dioxolane ring can be prepared in a similar manner using glycolic acid; glycoaldehyde (dimeric form); glycoaldehyde dialkylacetal such as diethylacetal; activated and/or protected glycolic acid or glycoaldehyde; or any other chemical equivalent of glycolic acid or glycoaldehyde. In a particular embodiment, the 1,3-dioxolane ring is formed using trimethylsilyl(trimethylsilyl)-acetate.

β-D or β-L-nucleosides can be manufactured by condensation of silylated purine or pyrimidine base with a 1,3-oxathiolane or 1,3-dioxolane intermediate. U.S. Pat. No. 5,204,466 discloses a method to condense a 1,3-oxathiolane with a silylated pyrimidine using tin chloride as a Lewis acid, which provides virtually complete β-stereoselectivity (see also Choi et al., loc. cit.). A number of U.S. patents disclose a process for the preparation of 1,3-oxathiolane nucleosides via condensation of a 1,3-oxathiolane 2-carboxylic acid ester with a protected silylated base in the presence of a silicon-based Lewis acid, followed by reduction of the ester to the corresponding hydroxymethyl group to afford the final product (see U.S. Pat. Nos. 5,663,320, 5,693,787, 5,696,254, 5,744,596, 5,756,706, 5,864,164).

U.S. Pat. No. 5,272,151 discloses a process using a 2-O-protected-5-O-acylated-1,3-oxathiolane for the preparation of nucleosides by condensation with a silylated purine or pyrimidine base in the presence of a titanium catalyst.

U.S. Pat. No. 6,215,004 discloses a process for producing 1,3-oxathiolane nucleosides that includes condensing 2-O-protected-methyl-5-chloro-1,3-oxathiolane with a silylated 5-fluorocytosine without a Lewis acid catalyst.

In a similar manner, the synthesis of biologically active compounds, such as mescarine (Hopkins M. H. et al., J. Am. Chem. Soc., 1991, 113, 5354), oxetanocin (Hambalek R. & Just J., Tetrahedron Lett., 1990, 31, 5445), kallolide A (Marshall J. A. et al J. Org. Chem., 1998, 63, 5962), (±)-kumausallene and (+)-epi-kumausallene (Grese T. S. et al., J. Org. Chem., 1993, 58, 2468) can be achieved using α-acyloxyacetaldehyde as a precursor.

The following working examples provide a further understanding of the process of manufacture of the present invention. These examples are of illustrative purpose, and are not meant to limit the scope of the invention. Equivalent, similar, or suitable solvents, reagents or reaction conditions may be substituted for those particular solvents, reagents or reaction conditions described herein without departing from the general scope of the process.

### EXAMPLES

Melting points were determined on a Mel-temp II laboratory device and are uncorrected. Nuclear magnetic resonance spectra were recorded on a Bruker 250 and AMX400 400 MHz spectrometers with tetramethykilane as the internal reference; chemical shifts' (δ) are reported in parts per million (ppm), and-the signals are described as s (singlet) d (doublet), t (triplet), q (quartet), bs (broad singlet), dd (doublet of doublet), and m (multiplet). UV spectra were obtained on a Beckman DU 650 spectrophotometer. Optical rotations were measured on a Jasco DIP-370 Digital Polarimeter. Mass spectra were measured using a Micromass Inc. Autospec High Resolution double focusing sector (EBE) MS spectrometers. Infrared spectra were recorded on a Nicolet 510 FT-IR spectrometer. Elemental analyses were performed by Atlantic Microlab, Inc., Norcross, Ga. All reactions were monitored using thin layer chromatography on Analtech, 200 mm silica gel GF plates. Dry 1,2-dichloroethane, dichloromethane, and acetonitrile were obtained by distillation from CaH₂ prior to use. Dry THF was obtained by distillation from Na and benzophenone when the solution became purple.

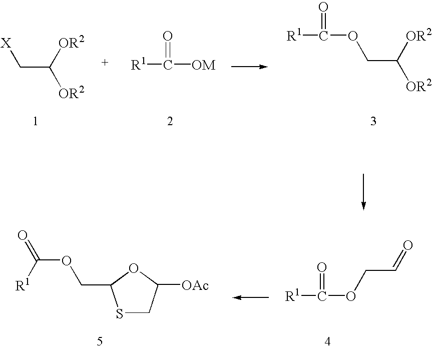

### Example 1

Preparation of Benzoyloxyacetaldehyde Diethyl Acetal (3)

NaOBz was added (2, R¹=Ph, M=Na) (0.055 mol, 7.9 g) to a solution of bromo acetaldehyde diethyl acetal (1, R²=Et) (0.1 mol, 19.7 g, 15.0 mL) in DMF (150 mL) and the mixture was refluxed for 2 h. Additional NaOBz (0.055 mol, 7.9 g) was charged portion-wise while refluxing. Reflux was continued for a total of 5 h, and then the mixture was allowed to cool to room temperature. Water (150 mL) was added, and the mixture was extracted with EtOAc (4×50 mL). The combined extracts were washed with water (4×25 mL), dried (Na₂SO₄), and concentrated in vacuo. The residue was dried azeotropically with toluene (2×20 mL) to give benzoyloxyacetaldehyde diethyl acetal 3 (R¹=Ph, R²=Et) as a dark oily product (21.45 g, 90%). This product was used directly in the next step without purification.

In a similar manner but using the corresponding sodium carboxylate, the following illustrative o-acyloxyacetaldehyde diethyl acetals are prepared:

acetoxyacetaldehyde diethyl acetal,

n-propionyloxyacetaldehyde diethyl acetal,

i-propionyloxyacetaldehyde diethyl acetal,

n-butyryloxyacetaldehyde diethyl acetal,

sec-butyryloxyacetaldehyde diethyl acetal,

t-butyryloxyacetaldehyde diethyl acetal,

valeroyloxyacetaldehyde diethyl acetal,

caproyloxyacetaldehyde diethyl acetal,

capriloyloxyacetaldehyde diethyl acetal,

benzoyloxyacetaldehyde diethyl acetal,

p-toluoyloxyacetaldehyde diethyl acetal,

m-toluoyloxyacetaldehyde diethyl acetal,

o-toluoyloxyacetaldehyde diethyl acetal,

p-chlorobenzoyloxyacetaldehyde diethyl acetal,

m-chlorobenzoyloxyacetaldehyde diethyl acetal,

o-chlorombenzoyloxyacetaldehyde diethyl acetal,

p-bromobenzoyloxyacetaldehyde diethyl acetal,

m-bromobenzoyloxyacetaldehyde diethyl acetal,

o-bromobenzoyloxyacetaldehyde diethyl acetal,

p-methoxybenzoyloxyacetaldehyde diethyl acetal,

m-methoxybenzoyloxyacetaldehyde diethyl acetal,

o-methoxybenzoyloxyacetaldehyde diethyl acetal,

p-nitrobenzoyloxyacetaldehyde diethyl acetal,

m-nitrobenzoyloxyacetaldehyde diethyl acetal,

o-nitrobenzoyloxyacetaldehyde diethyl acetal,

O-acetylsalicyloyloxyacetaldehyde diethyl acetal.

In a similar manner but using the corresponding dimethyl acetal, the following α-acyloxyacetaldehyde acetals are prepared:

acetoxyacetaldehyde dimethyl acetal,

n-propionyloxyacetaldehyde dimethyl acetal,

i-propionyloxyacetaldehyde dimethyl acetal,

n-butyryloxyacetaldehyde dimethyl acetal,

sec-butyryloxyacetaldehyde dimethyl acetal,

t-butyryloxyacetaldehyde dimethyl acetal,

valeroyloxyacetaldehyde dimethyl acetal,

caproyloxyacetaldehyde dimethyl acetal,

capriloyloxyacetaldehyde dimethyl acetal,

benzoyloxyacetaldehyde dimethyl acetal,

p-toluoyloxyacetaldehyde dimethyl acetal,

m-toluoyloxyacetaldehyde dimethyl acetal,

o-toluoyloxyacetaldehyde dimethyl acetal,

p-chlorobenzoyloxyacetaldehyde dimethyl acetal,

m-chlorobenzoyloxyacetaldehyde dimethyl acetal,

o-chlorobenzoyloxyacetaldehyde dimethyl acetal,

p-bromobenzoyloxyacetaldehyde dimethyl acetal,

m-bromobenzoyloxyacetaldehyde dimethyl acetal,

o-bromobenzoyloxyacetaldehyde dimethyl acetal,

p-methoxybenzoyloxyacetaldehyde dimethyl acetal,

m-methoxybenzoyloxyacetaldehyde dimethyl acetal,

o-methoxybenzoyloxyacetaldehyde dimethyl acetal,

p-nitrobenzoyloxyacetaldehyde dimethyl acetal,

m-nitrobenzoyloxyacetaldehyde dimethyl acetal,

o-nitrobenzoyloxyacetaldehyde dimethyl acetal,

O-acetylsalicyloyloxyacetaldehyde dimethyl acetal.

In a similar manner but using the corresponding dibenzyl acetal, the following α-acyloxyacetaldehyde acetals are prepared:

acetoxyacetaldehyde dibenzyl acetal,

n-propionyloxyacetaldehyde dibenzyl acetal,

i-propionyloxyacetaldehyde dibenzyl acetal,

n-butyryloxyacetaldehyde dibenzyl acetal,

sec-butyryloxyacetaldehyde dibenzyl acetal,

t-butyryloxyacetaldehyde dibenzyl acetal,

valeroyloxyacetaldehyde dibenzyl acetal,

caproyloxyacetaldehyde dibenzyl acetal,

capriloyloxyacetaldehyde dibenzyl acetal,

benzoyloxyacetaldehyde dibenzyl acetal,

p-toluoyloxyacetaldehyde dibenzyl acetal,

m-toluoyloxyacetaldehyde dibenzyl acetal,

o-toluoyloxyacetaldehyde dibenzyl acetal,

p-chlorobenzoyloxyacetaldehyde dibenzyl acetal,

m-chlorobenzoyloxyacetaldehyde dibenzyl acetal,

o-chlorobenzoyloxyacetaldehyde dibenzyl acetal,

p-bromobenzoyloxyacetaldehyde dibenzyl acetal,

m-bromobenzoyloxyacetaldehyde dibenzyl acetal,

o-bromobenzoyloxyacetaldehyde dibenzyl acetal,

p-methoxybenzoyloxyacetaldehyde dibenzyl acetal,

m-methoxybenzoyloxyacetaldehyde dibenzyl acetal,

o-methoxybenzoyloxyacetaldehyde dibenzyl acetal,

p-nitrobenzoyloxyacetaldehyde dibenzyl acetal,

m-nitrobenzoyloxyacetaldehyde dibenzyl acetal,

o-nitrobenzoyloxyacetaldehyde dibenzyl acetal,

O-acetylsalicyloyloxyacetaldehyde dibenzyl acetal.

In a similar manner but using the corresponding dineopentyl acetal, the following α-acyloxyacetaldehyde acetals are prepared:

acetoxyacetaldehyde dineopentyl acetal,

n-propionyloxyacetaldehyde dineopentyl acetal,

i-propionyloxyacetaldehyde dineopentyl acetal,

n-butyryloxyacetaldehyde dineopentyl acetal,

sec-butyryloxyacetaldehyde dineopentyl acetal,

t-butyryloxyacetaldehyde dineopentyl acetal,

valeroyloxyacetaldehyde dineopentyl acetal,

caproyloxyacetaldehyde dineopentyl acetal,

capriloyloxyacetaldehyde dineopentyl acetal,

benzoyloxyacetaldehyde dineopentyl acetal,

p-toluoyloxyacetaldehyde dineopentyl acetal,

m-toluoyloxyacetaldehyde dineopentyl acetal,

o-toluoyloxyacetaldehyde dineopentyl acetal,

p-chlorobenzoyloxyacetaldehyde dineopentyl acetal,

m-chlorobenzoyloxyacetaldehyde dineopentyl acetal,

o-chlorobenzoyloxyacetaldehyde dineopentyl acetal,

p-bromobenzoyloxyacetaldehyde dineopentyl acetal,

m-bromobenzoyloxyacetaldehyde dineopentyl acetal,

o-bromobenzoyloxyacetaldehyde dineopentyl acetal,

p-methoxybenzoyloxyacetaldehyde dineopentyl acetal,

m-methoxybenzoyloxyacetaldehyde dineopentyl acetal,

o-methoxybenzoyloxyacetaldehyde dineopentyl acetal,

p-nitrobenzoyloxyacetaldehyde dineopentyl acetal,

m-nitrobenzoyloxyacetaldehyde dineopentyl acetal,

o-nitrobenzoyloxyacetaldehyde dineopentyl acetal,

Salicyloyloxyacetaldehyde dineopentyl acetal.

In a similar manner but using the corresponding dimentyl acetal, the following α-acyloxyacetaldehyde acetals are prepared:

acetoxyacetaldehyde dimentyl acetal,

n-propionyloxyacetaldehyde dimentyl acetal,

i-propionyloxyacetaldehyde dimentyl acetal,

n-butyryloxyacetaldehyde dimentyl acetal,

sec-butyryloxyacetaldehyde dimentyl acetal,

t-butyryloxyacetaldehyde dimentyl acetal,

valeroyloxyacetaldehyde dimentyl acetal,

caproyloxyacetaldehyde dimentyl acetal,

capriloyloxyacetaldehyde dimentyl acetal,

benzoyloxyacetaldehyde dimentyl acetal,

p-toluoyloxyacetaldehyde dimentyl acetal,

m-toluoyloxyacetaldehyde dimentyl acetal,

o-toluoyloxyacetaldehyde dimentyl acetal,

p-chlorobenzoyloxyacetaldehyde dimentyl acetal,

m-chlorobenzoyloxyacetaldehyde dimentyl acetal,

o-chlorobenzoyloxyacetaldehyde dimentyl acetal,

p-bromobenzoyloxyacetaldehyde dimentyl acetal,

m-bromobenzoyloxyacetaldehyde dimentyl acetal,

o-bromobenzoyloxyacetaldehyde dimentyl acetal,

p-methoxybenzoyloxyacetaldehyde dimentyl acetal,

m-methoxybenzoyloxyacetaldehyde dimentyl acetal,

o-methoxybenzoyloxyacetaldehyde dimentyl acetal,

p-nitrobenzoyloxyacetaldehyde dimentyl acetal,

m-nitrobenzoyloxyacetaldehyde dimentyl acetal,

o-nitrobenzoyloxyacetaldehyde dimentyl acetal,

O-acetylsalicyloyloxyacetaldehyde dimentyl acetal.

### Example 2

Hydrolysis of Acetal (3) to Aldehyde (4)

A solution of acetal 3 (R¹=Ph, R²=Et) 2.38 g, 10 mmol) in aqueous formic acid (HCO₂H/H₂O=8/2 v/v, 24 mL) was stirred at room temperature for 3 h, and concentrated to dryness under reduced pressure (aspirator). The residue was co-evaporated with toluene (2×10 mL) to give aldehyde 4 (R¹=Ph).

Alternatively, when acetal 3 (R¹=Ph, R²=Et, 2.38 g) was treated with a mixture of trifluoroacetic acid (11 mL), THF (10 mL) and water (3 mL) for 3 h at room temperature, followed by evaporation of the solvent, the same aldehyde 4 (R¹=Ph) was obtained. This aldehyde was directly used in the next reaction without purification.

In a similar manner but using the corresponding sodium carboxylate, the following α-acyloxyacetaldehyde diethyl acetals are prepared:

acetoxyacetaldehyde,

n-propionyloxyacetaldehyde,

i-propionyloxyacetaldehyde,

n-butyryloxyacetaldehyde,

sec-butyryloxyacetaldehyde,

t-butyryloxyacetaldehyde,

valeroyloxyacetaldehyde,

caproyloxyacetaldehyde,

capriloyloxyacetaldehyde,

benzoyloxyacetaldehyde,

p-toluoyloxyacetaldehyde,

m-toluoyloxyacetaldehyde,

o-toluoyloxyacetaldehyde

p-chlorobenzoyloxyacetaldehyde

m-chlorobenzoyloxyacetaldehyde

o-chlorobenzoyloxyacetaldehyde

p-bromobenzoyloxyacetaldehyde,

m-bromobenzoyloxyacetaldehyde,

o-bromobenzoyloxyacetaldehyde,

p-methoxybenzoyloxyacetaldehyde,

m-methoxybenzoyloxyacetaldehyde,

o-methoxybenzoyloxyacetaldehyde,

p-nitrobenzoyloxyacetaldehyde,

m-nitrobenzoyloxyacetaldehyde,

o-nitrobenzoyloxyacetaldehyde,

O-acetylsalicyloyloxyacetaldehyde.

### Example 3

Cyclization and Acetylation

To a solution of the above aldehyde 4 in anhydrous THF (24 mL) were added dithiane-2,5-diol (0.912 g, 6 mmol) and BF₃.Et₂O (4.8 mmol, 0.64 mL, the amount of catalyst can be reduced) and the mixture was stirred at room temperature for 2 h. Solid was removed by filtration. The following were added to the filtrate: pyridine (28.8 mmol, 2.3 g, 2.3 mL), acetic anhydride (15 mmol, 1.42 mL) and 4-dimethylaminopyridine (1 mmol, 122 mg). The mixture was then stirred at room temperature for 16 h. Solvent was removed and the residue was dissolved in EtOAc (100 mL). The mixture was washed with water (3×10 mL) and dried (Na₂SO₄). Solvent was removed and the residue was purified by silica gel column chromatography (20% of EtOAc in hexanes) to give racemic 5-acetoxy-2-(benzoyloxy)methyl-1,3-oxathiolane 5 (R¹=Ph) as an oil. The process yielded 2.2 g, 78% overall, in three steps.

In a similar manner but using the corresponding acyloxyacetaldehydes, the following 2-(acyloxy)methyl-5-acetoxy-1,3-oxathiolanes are prepared:

5-acetoxy-2-(acetoxy)methyl-1,3-oxathiolane,

5-acetoxy-2-(n-propionyloxy)methyl-1,3-oxathiolane,

5-acetoxy-2-(i-propionyloxy)methyl-1,3-oxathiolane,

5-acetoxy-2-(n-butyryloxy)methyl-1,3-oxathiolane,

5-acetoxy-2-(sec-butyryloxy)methyl-1,3-oxathiolane,

5-acetoxy-2-(t-butyryloxy)methyl-1,3-oxathiolane,

5-acetoxy-2-valeroyloxymethyl-1,3-oxathiolane,

5-acetoxy-2-caproyloxymethyl-1,3-oxathiolane,

5-acetoxy-2-(capriloyloxy)methyl-1,3-oxathiolane,

5-acetoxy-2-benzoyloxymethyl-1,3-oxathiolane,

5-acetoxy-2-(p-toluoyloxy)methyl-1,3-oxathiolane,

5-acetoxy-2-(m-toluoyloxy)methyl-1,3-oxathiolane,

5-acetoxy-2-(o-toluoyloxy)methyl-1,3-oxathiolane,

5-acetoxy-2-(p-chlorobenzoyloxy)methyl-1,3-oxathiolane,

5-acetoxy-2-(m-chlorobenzoyloxy)methyl-1,3-oxathiolane,

5-acetoxy-2-(o-chlorobenzoyloxy)methyl-1,3-oxathiolane,

5-acetoxy-2-(p-bromobenzoyloxy)methyl-1,3-oxathiolane,

5-acetoxy-2-(m-bromobenzoyloxy)methyl-1,3-oxathiolane,

5-acetoxy-2-(o-bromobenzoyloxy)methyl-1,3-oxathiolane,

5-acetoxy-2-(p-methoxybenzoyloxy)methyl-1,3-oxathiolane,

5-acetoxy-2-(m-methoxybenzoyloxy)methyl-1,3-oxathiolane,

5-acetoxy-2-(o-methoxybenzoyloxy)methyl-1,3-oxathiolane,

5-acetoxy-2-(p-nitrobenzoyloxy)methyl-1,3-oxathiolane,

5-acetoxy-2-(m-nitrobenzoyloxy)methyl-1,3-oxathiolane,

5-acetoxy-2-(o-nitrobenzoyloxy)methyl-1,3-oxathiolane,

5-acetoxy-2-(O-acetylsalicyloxy)methyl-1,3-oxathiolane.

We claim:

1. A process for the manufacture of an α-acyloxyacetaldehyde of the formula: 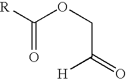
wherein R is hydrogen, alkyl, alkenyl, alkynyl, or aryl, that can be optionally substituted with one or more substituents that do not otherwise adversely affect the reaction process and wherein the R can be a chiral moiety; that includes the steps of:
a) reacting a 2,2-dialkoxyethyl halide of formula: 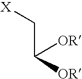
wherein X is a halide or a suitable leaving group;
and each R′ is independently an alkyl, alkenyl, alkynyl, aryl, aralkyl, heteroaryl, or heterocycle;
with an appropriate carboxylate of formula ⁻OC(═O)R, wherein R is hydrogen, alkyl, alkenyl, alkynyl, or aryl, that can be optionally substituted with one or more substituents;
to obtain an acetal of the formula 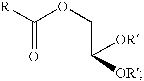
and
b) hydrolyzing the acetal to form the α-acyloxyacetaldehyde.

2. The process according to claim 2, wherein the acetal is an α-acyloxyacetaldehyde dialkyl acetal.

3. The process according to claim 2, wherein the a-acyloxyacetaldehyde dialkyl acetal is an α-acyloxyacetaldehyde diethyl acetal.

4. The process according to claim 3, wherein the α-acyloxyacetaldehyde diethyl acetal is selected from the group consisting of
n-propionyloxyacetaldehyde diethyl acetal,
i-propionyloxyacetaldehyde diethyl acetal,
n-butyryloxyacetaldehyde diethyl acetal,
sec-butyryloxyacetaldehyde diethyl acetal,
t-butyryloxyacetaldehyde diethyl acetal,
valeroyloxyacetaldehyde diethyl acetal,
caproyloxyacetaldehyde diethyl acetal,
capriloyloxyacetaldehyde diethyl acetal,
p-toluoyloxyacetaldehyde diethyl acetal,
m-toluoyloxyacetaldehyde diethyl acetal,
o-toluoyloxyacetaldehyde diethyl acetal,
p-chlorobenzoyloxyacetaldehyde diethyl acetal,
m-chlorobenzoyloxyacetaldehyde diethyl acetal,
o-chlorobenzoyloxyacetaldehyde diethyl acetal,
p-bromobenzoyloxyacetaldehyde diethyl acetal,
m-bromobenzoyloxyacetaldehyde diethyl acetal,
o-bromobenzoyloxyacetaldehyde diethyl acetal,
p-methoxybenzoyloxyacetaldehyde diethyl acetal,
m-methoxybenzoyloxyacetaldehyde diethyl acetal,
o-methoxybenzoyloxyacetaldehyde diethyl acetal,
p-nitrobenzoyloxyacetaldehyde diethyl acetal,
m-nitrobenzoyloxyacetaldehyde diethyl acetal,
o-nitrobenzoyloxyacetaldehyde diethyl acetal, and
O-acetylsalicyloyloxyacetaldehyde diethyl acetal.

5. The process of claim 2, wherein the α-acyloxyacetaldehyde dialkyl acetal is an α-acyloxyacetaldehyde dimethyl acetal.

6. The process of claim 5, wherein the α-acyloxyacetaldehyde dimethyl acetal is selected from the group consisting of
acetoxyacetaldehyde dimethyl acetal,
n-propionyloxyacetaldehyde dimethyl acetal,
i-propionyloxyacetaldehyde dimethyl acetal,
n-butyryloxyacetaldehyde dimethyl acetal,
sec-butyryloxyacetaldehyde dimethyl acetal,
t-butyryloxyacetaldehyde dimethyl acetal,
valeroyloxyacetaldehyde dimethyl acetal,
caproyloxyacetaldehyde dimethyl acetal,
capriloyloxyacetaldehyde dimethyl acetal,
benzoyloxyacetaldehyde dimethyl acetal,
p-toluoyloxyacetaldehyde dimethyl acetal,
m-toluoyloxyacetaldehyde dimethyl acetal,
o-toluoyloxyacetaldehyde dimethyl acetal,
p-chlorobenzoyloxyacetaldehyde dimethyl acetal,
m-chlorobenzoyloxyacetaldehyde dimethyl acetal,
o-chlorobenzoyloxyacetaldehyde dimethyl acetal,
p-bromobenzoyloxyacetaldehyde dimethyl acetal,
m-bromobenzoyloxyacetaldehyde dimethyl acetal,
o-bromobenzoyloxyacetaldehyde dimethyl acetal,
p-methoxybenzoyloxyacetaldehyde dimethyl acetal,
m-methoxybenzoyloxyacetaldehyde dimethyl acetal,
o-methoxybenzoyloxyacetaldehyde dimethyl acetal,
p-nitrobenzoyloxyacetaldehyde dimethyl acetal,
m-nitrobenzoyloxyacetaldehyde dimethyl acetal,
o-nitrobenzoyloxyacetaldehyde dimethyl acetal, and
O-acetylsalicyloyloxyacetaldehyde dimethyl acetal.

7. The process of claim 2, wherein the α-acyloxyacetaldehyde dialkyl acetal is selected from the group consisting of:
acetoxyacetaldehyde dineopentyl acetal,
n-propionyloxyacetaldehyde dineopentyl acetal,
i-propionyloxyacetaldehyde dineopentyl acetal,
n-butyryloxyacetaldehyde dineopentyl acetal,
sec-butyryloxyacetaldehyde dineopentyl acetal,
t-butyryloxyacetaldehyde dineopentyl acetal,
valeroyloxyacetaldehyde dineopentyl acetal,
caproyloxyacetaldehyde dineopentyl acetal,
capriloyloxyacetaldehyde dineopentyl acetal,
benzoyloxyacetaldehyde dineopentyl acetal,
p-toluoyloxyacetaldehyde dineopentyl acetal,
m-toluoyloxyacetaldehyde dineopentyl acetal,
o-toluoyloxyacetaldehyde dineopentyl acetal,
p-chlorobenzoyloxyacetaldehyde dineopentyl acetal,
m-chlorobenzoyloxyacetaldehyde dineopentyl acetal,
o-chlorobenzoyloxyacetaldehyde dineopentyl acetal,
p-bromobenzoyloxyacetaldehyde dineopentyl acetal,
m-bromobenzoyloxyacetaldehyde dineopentyl acetal,
o-bromobenzoyloxyacetaldehyde dineopentyl acetal,
p-methoxybenzoyloxyacetaldehyde dineopentyl acetal,
m-methoxybenzoyloxyacetaldehyde dineopentyl acetal,
o-methoxybenzoyloxyacetaldehyde dineopentyl acetal,
p-nitrobenzoyloxyacetaldehyde dineopentyl acetal,
m-nitrobenzoyloxyacetaldehyde dineopentyl acetal,
o-nitrobenzoyloxyacetaldehyde dineopentyl acetal, and
salicyloyloxyacetaldehyde dineopentyl acetal.

8. The process of claim 1, wherein the acetal is an α-acyloxyacetaldehyde diaralkyl acetal.

9. The process of claim 8, wherein the α-acyloxyacetaldehyde diaralkyl acetal is selected from the group consisting of
acetoxyacetaldehyde dibenzyl acetal,
n-propionyloxyacetaldehyde dibenzyl acetal,
i-propionyloxyacetaldehyde dibenzyl acetal,
n-butyryloxyacetaldehyde dibenzyl acetal,
sec-butyryloxyacetaldehyde dibenzyl acetal,
t-butyryloxyacetaldehyde dibenzyl acetal,
valeroyloxyacetaldehyde dibenzyl acetal,
caproyloxyacetaldehyde dibenzyl acetal,
capriloyloxyacetaldehyde dibenzyl acetal,
benzoyloxyacetaldehyde dibenzyl acetal,
p-toluoyloxyacetaldehyde dibenzyl acetal,
m-toluoyloxyacetaldehyde dibenzyl acetal,
o-toluoyloxyacetaldehyde dibenzyl acetal,
p-chlorobenzoyloxyacetaldehyde dibenzyl acetal,
m-chlorobenzoyloxyacetaldehyde dibenzyl acetal,
o-chlorobenzoyloxyacetaldehyde dibenzyl acetal,
p-bromobenzoyloxyacetaldehyde dibenzyl acetal,
m-bromobenzoyloxyacetaldehyde dibenzyl acetal,
o-bromobenzoyloxyacetaldehyde dibenzyl acetal,
p-methoxybenzoyloxyacetaldehyde dibenzyl acetal,
m-methoxybenzoyloxyacetaldehyde dibenzyl acetal,
o-methoxybenzoyloxyacetaldehyde dibenzyl acetal,
p-nitrobenzoyloxyacetaldehyde dibenzyl acetal,
m-nitrobenzoyloxyacetaldehyde dibenzyl acetal,
o-nitrobenzoyloxyacetaldehyde dibenzyl acetal, and
O-acetylsalicyloyloxyacetaldehyde dibenzyl acetal.

10. The process of claim 1, wherein the acetal is an α-acyloxyacetaldehyde diterpenoid acetal.

11. The process of claim 10, wherein the α-acyloxyacetaldehyde diterpenoid acetal is selected from the group consisting of
acetoxyacetaldehyde dimentyl acetal,
n-propionyloxyacetaldehyde dimentyl acetal,
i-propionyloxyacetaldehyde dimentyl acetal,
n-butyryloxyacetaldehyde dimentyl acetal,
sec-butyryloxyacetaldehyde dimentyl acetal,
t-butyryloxyacetaldehyde dimentyl acetal,
valeroyloxyacetaldehyde dimentyl acetal,
caproyloxyacetaldehyde dimentyl acetal,
capriloyloxyacetaldehyde dimentyl acetal,
benzoyloxyacetaldehyde dimentyl acetal,
p-toluoyloxyacetaldehyde dimentyl acetal,
m-toluoyloxyacetaldehyde dimentyl acetal,
o-toluoyloxyacetaldehyde dimentyl acetal,
p-chlorobenzoyloxyacetaldehyde dimentyl acetal,
m-chlorobenzoyloxyacetaldehyde dimentyl acetal,
o-chlorobenzoyloxyacetaldehyde dimentyl acetal,
p-bromobenzoyloxyacetaldehyde dimentyl acetal,
m-bromobenzoyloxyacetaldehyde dimentyl acetal,
o-bromobenzoyloxyacetaldehyde dimentyl acetal,
p-methoxybenzoyloxyacetaldehyde dimentyl acetal,
m-methoxybenzoyloxyacetaldehyde dimentyl acetal,
o-methoxybenzoyloxyacetaldehyde dimentyl acetal,
p-nitrobenzoyloxyacetaldehyde dimentyl acetal,
m-nitrobenzoyloxyacetaldehyde dimentyl acetal,
o-nitrobenzoyloxyacetaldehyde dimentyl acetal, and
o-acetylsalicyloyloxyacetaldehyde dimentyl acetal.

12. The process of claim 1, wherein the α-acylacetaldehyde is selected from the group consisting of
acetoxyacetaldehyde,
n-propionyloxyacetaldehyde,
i-propionyloxyacetaldehyde,
n-butyryloxyacetaldehyde,
sec-butyryloxyacetaldehyde,
t-butyryloxyacetaldehyde,
valeroyloxyacetaldehyde,
caproyloxyacetaldehyde,
capriloyloxyacetaldehyde,
benzoyloxyacetaldehyde,
p-toluoyloxyacetaldehyde,
m-toluoyloxyacetaldehyde,
o-toluoyloxyacetaldehyde,
p-chlorobenzoyloxyacetaldehyde
m-chlorobenzoyloxyacetaldehyde
o-chlorobenzoyloxyacetaldehyde
p-bromobenzoyloxyacetaldehyde,
m-bromobenzoyloxyacetaldehyde,
o-bromobenzoyloxyacetaldehyde,
p-methoxybenzoyloxyacetaldehyde,
m-methoxybenzoyloxyacetaldehyde,
o-methoxybenzoyloxyacetaldehyde,
p-nitrobenzoyloxyacetaldehyde,
m-nitrobenzoyloxyacetaldehyde,
o-nitrobenzoyloxyacetaldehyde, and
o-acetylsalicyloyloxyacetaldehyde.

13. A process for the manufacture of a 1,3-oxathiolane of the formula: 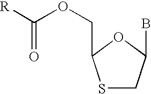
wherein R is hydrogen, alkyl, alkenyl, alkynyl, or aryl, that can be optionally substituted with one or more substituents that do not otherwise adversely affect the reaction process and wherein R can be a chiral moiety; and B is a purine or pyrimidine base; that includes the steps of:
a) preparing α-acyloxyacetaldehyde according to the process of claim 1, and then reacting with mercaptoacetic acid, mercaptoaldehyde, or mercaptoacetaldehyde dialkacetal to form an intermediate 1,3-oxathiolane of the formula: 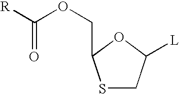
wherein R is hydrogen, alkyl, alkenyl, alkynyl, or aryl, that can be optionally substituted with one or more substituents that do not otherwise adversely affect the reaction process and wherein the R can be a chiral moiety; and L is a leaving group; and
b) coupling the intermediate 1,3-oxathiolane with a purine or pyrimidine base in the presence of a Lewis acid to obtain the 1,3-oxathiolane.

14. The process according to claim 13, wherein the leaving group selected from the group consisting of O-acyl, O-alkyl, O-tosylate, O-mesylate, and halogen (F, Cl, Br, I).

15. The process according to claim 13, wherein the Lewis acid is selected from the group consisting of TMSCl, TMSI, TMSTf, SnCl₄, and TiCl₄.

16. A process for the manufacture of a 1,3-dioxolane of the formula: 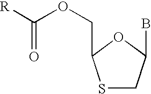
wherein R is hydrogen, alkyl, alkenyl, alkynyl, or aryl, that can be optionally substituted with one or more substituents that do not otherwise adversely affect the reaction process and wherein the R can be a chiral moiety; and B is a purine or pyrimidine base; comprising the steps of:
a) preparing α-acyloxyacetaldehyde according to the process of claim 1 and then reacting it with glycolic acid, glycoaldehyde, or glycoaldehyde dialkylacetal to form an intermediate 1,3-dioxolane of the formula: 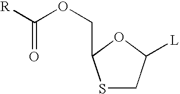
wherein R is hydrogen, alkyl, alkenyl, alkynyl, or aryl, that can be optionally substituted with one or more substituents that do not otherwise adversely affect the reaction process and wherein the R can be a chiral moiety; and L is a leaving group;
b) coupling the intermediate 1,3-dioxolane with a purine or pyrimidine base in the presence of a Lewis acid to obtain the 1,3-dioxolane nucleoside.

17. The process according to claim 16, wherein the leaving group selected from the group consisting of O-acyl, O-alkyl, O-tosylate, O-mesylate, and halogen.

18. The process according to claim 17, wherein the Lewis acid is selected from the group consisting of TMSCl, TMSI, TMSTf, SnCl₄, and TiCl₄.

19. The process of claims 1, wherein the hydrolysis of the acetal is carried out with an organic acid.

20. The process of claim 19, the organic acid is aqueous formic acid.

21. The process of claim 13, where the intermediate 1,3-oxathiolane is selected from the group consisting of:
5-acetoxy-2-(acetoxy)methyl-1,3-oxathiolane,
5-acetoxy-2-(n-propionyloxy)methyl-1,3-oxathiolane,
5-acetoxy-2-(i-propionyloxy)methyl-1,3-oxathiolane,
5-acetoxy-2-(n-butyryloxy)methyl-1,3-oxathiolane,
5-acetoxy-2-(sec-butyryloxy)methyl-1,3-oxathiolane,
5-acetoxy-2-(t-butyryloxy)methyl-1,3-oxathiolane,
5-acetoxy-2-valeroyloxymethyl-1,3-oxathiolane,
5-acetoxy-2-caproyloxymethyl-1,3-oxathiolane,
5-acetoxy-2-(capriloyloxy)methyl-1,3-oxathiolane,
5-acetoxy-2-benzoyloxymethyl-1,3-oxathiolane,
5-acetoxy-2-(p-toluoyloxy)methyl-1,3-oxathiolane,
5-acetoxy-2-(m-toluoyloxy)methyl-1,3-oxathiolane,
5-acetoxy-2-(o-toluoyloxy)methyl-1,3-oxathiolane,
5-acetoxy-2-(p-chlorobenzoyloxy)methyl-1,3-oxathiolane,
5-acetoxy-2-(m-chlorobenzoyloxy)methyl-1,3-oxathiolane,
5-acetoxy-2-(o-chlorobenzoyloxy)methyl 1,3-oxathiolane,
5-acetoxy-2-(p-bromobenzoyloxy)methyl-1,3-oxathiolane,
5-acetoxy-2-(m-bromobenzoyloxy)methyl-1,3-oxathiolane,
5-acetoxy-2-(o-bromobenzoyloxy)methyl-1,3-oxathiolane,
5-acetoxy-2-(p-methoxybenzoyloxy)methyl-1,3-oxathiolane,
5-acetoxy-2-(methoxybenzoyloxy)methyl-1,3-oxathiolane,
5-acetoxy-2-(o-methoxybenzoyloxy)methyl-1,3-oxathiolane,
5-acetoxy-2-(p-nitrobenzoyloxy)methyl-1,3-oxathiolane,
5-acetoxy-2-(m-nitrobenzoyloxy)methyl-1,3-oxathiolane,
5-acetoxy-2-(o-nitrobenzoyloxy)methyl-1,3-oxathiolane, and
5-acetoxy-2-(O-acetylsalicyloxy)methyl-1,3-oxathiolane.

22. The process of claim 13 or 16, wherein the pyrimidine base is selected from cytosine and 5-fluorocytosine.
